(19) United States
(12) Patent Application Publication
(10) Pub. No.: US 2022/0061807 A1
(43) Pub. Date: Mar. 3, 2022

(54) ACTIVELY DAMPED ULTRASONIC TRANSDUCER

(71) Applicant: UNIVERSITY OF SOUTHERN CALIFORNIA, Los Angeles, CA (US)

(72) Inventor: Jesse Tong-Pin Yen, La Crescenta, CA (US)

(21) Appl. No.: 17/412,696

(22) Filed: Aug. 26, 2021

Related U.S. Application Data
(60) Provisional application No. 63/070,742, filed on Aug. 26, 2020.

(51) Int. Cl. A61B 8/00

(52) U.S. Cl. CPC A61B 8/4483; B06B 1/0644; A61B 8/469; A61B 8/463

(57) ABSTRACT

An ultrasound system is disclosed that utilizes an arbitrary waveform generator, memory, and an ultrasound transducer. A plurality of excitation waveforms are stored in the memory and may be output from the arbitrary waveform generator to an ultrasound transducer. At least one first excitation waveform is stored in the memory and includes an excitation portion with no damping portion (e.g., for one ultrasound procedure; such that an output from the ultrasound transducer is of a first bandwidth). At least one second excitation waveform is stored in the memory and includes an excitation portion and a damping portion (e.g., for another ultrasound procedure; such that an output from the ultrasound transducer is of a second bandwidth that is larger than the first bandwidth).

### CROSS-REFERENCE TO RELATED APPLICATIONS

This patent application is a non-provisional patent application of, and claims the benefit of, co-pending U.S. Provisional Patent Application Ser. No. 63/070,742, that is entitled “ACTIVELY DAMPED ULTRASONIC TRANSDUCER,” that was filed on 26-Aug.-2020, and the entire disclosure of which is hereby incorporated by reference herein.

### GOVERNMENT LICENSE RIGHTS

This invention was made with United States government support under Contract No. R21HL132257 by the National Institutes of Health. The United States government has certain rights in this invention.

### FIELD

The present disclosure generally relates to the field of ultrasound imaging and, more particularly, controlling a bandwidth that is output by a transducer of an ultrasound imaging system.

### BACKGROUND

Ultrasound techniques such as ultrasound-guided high-intensity focused ultrasound (HIFU) and acoustic radiation forced impulse (ARFI) require transducers capable of mid-to high-power with narrow bandwidth, while imaging requires transducers with low-power and broad bandwidth. Oftentimes, separate transducers are used for imaging and therapy.

Short, broadband pulses are desirable for ultrasound imaging. Pulses may be achieved by appropriately designing the acoustic backing and front layers of an ultrasound transducer. This can be referred to as passive damping. However, backing and matching layers of an ultrasonic transducer can be difficult to fabricate.

Technologies such as focused ultrasound for therapeutic applications, acoustic radiation force imaging, harmonic imaging, and the like have placed demanding and oftentimes conflicting design requirements on ultrasound transducers. For applications such as high-intensity focused ultrasound (HIFU), a low impedance or air backing is often used to minimize energy losses and avoid overheating. Ultrasound-guided HIFU uses a HIFU transducer with an inner circle removed for placement of the imaging transducer. This may limit the field of view. For acoustic radiation force imaging (ARFI), higher transmit power is desired while still maintaining broad bandwidth.

In ultrasound imaging, damping or shortening of the emitted pulse is necessary to achieve fine axial resolution. For transducers using piezoceramics, this can be achieved mechanically or electrically. Mechanically or acoustically, the use of a backing material with acoustic impedance in the range of 3-7 MRayls and at least one matching layer can result in adequate bandwidth and efficiency. A backing with a higher acoustic impedance will minimize the reflections between the back face of the piezoelectric material and the backing, but reduces the total energy emitted to the front medium. Backings are usually lossy or highly absorptive to minimize echoes returning from the opposite end of the ceramic and backing boundary. Use of multiple quarter wave matching layers can also improve the efficiency and bandwidth. Passive electrical tuning strategies can also increase transducer bandwidth. Array elements may have an impedance on the order of several hundred Ohms. In transmit, optimal energy transfer is achieved when they array element is matched to the output impedance of the transmitter. Matching impedances requires the use of tuning elements near the transducer and/or towards the system end.

### SUMMARY

An ultrasound system is presented herein. Both the configuration of such an ultrasound system and the use/operation of such an ultrasound system are within the scope of this Summary.

An ultrasound system may include an arbitrary waveform generator, an ultrasound transducer that is operatively connected with this arbitrary waveform generator (directly or indirectly), and memory. This memory may store a plurality of excitation waveforms, each of which may be output (e.g., separately; for different ultrasound procedures) to the ultrasound transducer by the arbitrary waveform generator. The memory may include at least one first excitation waveform, with each first excitation waveform including an excitation portion but no damping portion. The memory may also include at least one second excitation waveform, with each second excitation waveform including an excitation portion and a damping portion.

Any appropriate ultrasound transducer may be utilized by the ultrasound system, such as an air-backed ultrasound transducer. The ultrasound transducer may exclude a backing layer, may or may not include one or more matching layers, or any combination thereof in view of the above-noted excitation waveforms stored in memory (e.g., a second excitation waveform that includes both an excitation portion and a damping portion).

In the case where there are a plurality of first excitation waveforms in memory, each of these first excitation waveforms may utilize a different excitation portion. In the case where there are a plurality of second excitation waveforms in memory, each of the second excitation waveforms may use a different excitation portion, a different damping portion, or a different combination of an excitation portion and damping portion.

Consider a case: 1) where a first excitation waveform (again, that does not use a damping portion) is used by the arbitrary waveform generator as a drive signal for the ultrasound transducer, and that outputs a first ultrasound signal from the ultrasound transducer of a first bandwidth; and 2) where a second excitation waveform (again, that uses both an excitation portion and a damping portion) is used by the arbitrary waveform generator as a drive signal for this same ultrasound transducer, and that in turn outputs a second ultrasound signal of a second bandwidth (e.g., the noted first excitation waveform may be used for a first ultrasound procedure and the noted second excitation waveform may be used for a different, second ultrasound procedure). The second bandwidth associated with the second excitation waveform (with a damping portion) is larger than the first bandwidth associated with the first excitation waveform (without a damping portion). The difference in bandwidths may be significant. For instance, the second bandwidth may be at least two times, three times, or at least four times larger than the first bandwidth. The ability to output different bandwidths from the same ultrasound transducer accommodates using the same ultrasound transducer for various different ultrasound procedures (e.g., therapy, imaging).

Various aspects of the present disclosure are also addressed by the following paragraphs and in the noted combinations:

1. A system comprising:

an ultrasonic transducer configured to transmit and receive ultrasonic acoustic waves; and

an arbitrary waveform generator configured to generate waveforms for dampening ringing from the ultrasonic transducer.

2. The system of paragraph 1, wherein the ultrasonic transducer is an air-backed transducer.

3. The system of paragraph 1, wherein the ultrasonic transducer is configured to provide mid-to high-power, narrow bandwidth waves or low-power, broad bandwidth waves.

4. The system of paragraph 1, wherein the waveforms generated by the arbitrary waveform generator include an excitation pulse and a corresponding dampening pulse.

5. The system of paragraph 4, wherein the dampening pulse is an inversion of the excitation pulse with reduced amplitude or more cycles are used in the dampening pulse than in the excitation pulse.

6. The system of paragraph 1, further comprising a power amplifier connected to the ultrasonic transducer and configured to receive the generate waveforms from the arbitrary waveform generator.

7. A method comprising:

transmitting ultrasonic acoustic waves using an ultrasonic transducer; and

using an arbitrary waveform generator to generate waveforms for dampening ringing from the ultrasonic transducer.

8. The method of paragraph 7, further comprising configuring the ultrasonic transducer to provide mid-to high-power, narrow bandwidth waves or low-power, broad bandwidth waves.

9. The method of paragraph 7, wherein generating the waveforms by the arbitrary waveform generator include generating an excitation pulse and a corresponding dampening pulse.

10. The method of paragraph 9, wherein the dampening pulse is an inversion of the excitation pulse with reduced amplitude.

11. A method of dampening an ultrasonic transducer comprising:

implementing a 1-D KLM transmission line model; and

generating arbitrary waveforms in the time domain, the arbitrary waveforms including an initial excitation pulse followed by damping pulses of varying amplitude which are out of phase with respect to the initial excitation pulse.

12. The method of paragraph 11, wherein the arbitrary waveforms are generated by adjusting an amplitude of each subsequent pulse or adjusting an amplitude of different portions of each subsequent pulse.

13. The method of paragraph 11, wherein the arbitrary waveforms are empirically and iteratively created.

14. The method of paragraph 11, further comprising optimizing bandwidth using at least one of a multidimensional unconstrained nonlinear minimization, genetic algorithms, or particle swarm optimization.

15. An ultrasound system, comprising:

an arbitrary waveform generator;

an ultrasound transducer operatively connected with said arbitrary waveform generator; and

memory comprising a plurality of excitation waveforms transmittable from said arbitrary waveform generator to said ultrasound transducer, wherein a first excitation waveform of said plurality of excitation waveforms comprises an excitation portion with no damping portion, and wherein a second excitation waveform of said plurality of excitation waveforms comprises an excitation portion and a damping portion.

16. The ultrasound system of paragraph 15, wherein said ultrasound transducer comprises an air-backed ultrasound transducer.

17. The ultrasound system of paragraph 16, wherein said ultrasound transducer further comprises a first matching layer.

18. The ultrasound system of any of paragraph 15-17, wherein said ultrasound transducer excludes a hacking layer.

19. The ultrasound system of any of paragraphs 15-18, further comprising a plurality of said first excitation waveforms.

20. The ultrasound system of paragraph 19, wherein each of said plurality of first excitation waveforms uses a different said excitation portion.

21. The ultrasound system of any of paragraphs 15-20, further comprising a plurality of said second excitation waveforms.

22. The ultrasound system of paragraph 21, wherein each of said plurality of second excitation waveforms uses a different said excitation portion, a different said damping portion, or a different combination thereof.

23. The ultrasound system of any of paragraphs 15-22, wherein said first excitation waveform output from said arbitrary waveform generator outputs a first ultrasound signal from said ultrasound transducer of a first bandwidth, wherein said second excitation waveform output from said arbitrary waveform generator outputs a second ultrasound signal from said ultrasound transducer of a second bandwidth, and wherein said second bandwidth is larger than said first bandwidth.

24. The ultrasound system of paragraph 23, wherein said second bandwidth is at least two times larger than said first bandwidth.

25. The ultrasound system of paragraph 23, wherein said second bandwidth is at least three times larger than said first bandwidth.

26. The ultrasound system of paragraph 23, wherein said second bandwidth is at least four times larger than said first bandwidth.

27. The ultrasound system of any of paragraphs 15-26, wherein said excitation portion of said second excitation waveform precedes said damping portion of said second excitation waveform.

28. The ultrasound system of any of paragraphs 15-27, wherein said damping portion of said second excitation waveform is at least one of inverted and of a reduced amplitude compared to said excitation portion of said second excitation waveform.

29. The ultrasound system of any of paragraphs 15-27, wherein said damping portion of said second excitation waveform comprises damping pulses of varying amplitude which are out of phase with said excitation portion of said second excitation waveform.

30. The ultrasound system of any of paragraphs 15-27, wherein said damping portion of said second excitation waveform comprises an inverted cycle pulse of a first cycle with a smaller first amplitude than said excitation portion, followed by an inverted cycle pulse of a second cycle with a smaller second amplitude than said excitation portion, and wherein said second amplitude is also smaller than said first amplitude.

31. The Ultrasound system of paragraph 30, wherein said first cycle is a 1.5 cycle pulse and said second cycle is a 2 cycle pulse.

32. The ultrasound system of any of paragraphs 15-27, wherein said dampening portion comprises a first dampening pulse of a first amplitude that is less than said excitation portion, and a second dampening pulse following said first dampening pulse that is of a second amplitude that is less than both said first amplitude and said excitation portion.

33. The ultrasound system of any of paragraphs 15-32, further comprising at least one of a user interface and a display.

34. A method of executing an ultrasound procedure using an ultrasound system comprising an arbitrary waveform generator, memory, and an ultrasound transducer, said method comprising:

selecting an excitation waveform from a plurality of excitation waveforms stored in said memory and that defines a selected excitation waveform, wherein a first excitation waveform of said plurality of excitation waveforms comprises an excitation portion with no damping portion, and wherein a second excitation waveform of said plurality of excitation waveforms comprises an excitation portion and a damping portion;

sending said selected excitation waveform from said arbitrary waveform generator to said ultrasound transducer; and

transmitting an ultrasound signal from said ultrasound transducer in response to said sending.

35. The method of paragraph 34, further comprising presenting said plurality of excitation waveforms on a display.

36. The method of paragraph 35, wherein said selecting comprises using a user interface to select one of said plurality of excitation waveforms presented on said display.

37. The method of any of paragraphs 35-36, wherein said presenting comprises presenting said plurality of excitation forms in two different groups, wherein a first group comprises a plurality of said first excitation waveforms, and wherein a second group comprises a plurality of said second excitation waveforms.

38. The method of any of paragraphs 35-37, wherein said selecting is based upon a bandwidth of said Ultrasound signal provided by said selected excitation waveform.

39. The method of any of paragraphs 35-38, wherein said selecting is based upon a target application for said ultrasound signal.

40. The method of paragraph 39, wherein said target application is selected from the group consisting of therapy and imaging.

41. The method of paragraph 35, wherein said ultrasound system is the ultrasound system of any of paragraphs 1-33.

42. A computer-readable storage medium, comprising:

a plurality of excitation waveforms, wherein a first excitation waveform of said plurality of excitation waveforms comprises an excitation portion with no damping portion, and wherein a second excitation waveform of said plurality of excitation waveforms comprises an excitation portion and a damping portion; and

a protocol configured to:
present at least some of said plurality of excitation waveforms on a display; and
allow for selection of any one of said plurality of excitation waveforms through a user interface.

43. The computer-readable storage medium of paragraph 42, wherein said protocol is further configured to allow an arbitrary waveform generator to transmit a selected one of said plurality of excitation waveforms to an ultrasound transducer.

44. The computer-readable storage medium of any of paragraphs 42-43, further comprising a plurality of said first excitation waveforms.

45. The computer-readable storage medium of paragraph 44, wherein each of said plurality of first excitation waveforms uses a different said excitation portion.

46. The computer-readable storage medium of any of paragraphs 42-45, further comprising a plurality of said second excitation waveforms.

47. The computer-readable storage medium of paragraph 46, wherein each of said plurality of second excitation waveforms uses a different said excitation portion, a different said damping portion, or a combination thereof.

48. The computer-readable storage medium of any of paragraphs 42-47, wherein said first excitation waveform is configured to output a first ultrasound signal from an ultrasound transducer of a first bandwidth, wherein said second excitation waveform output is configured to output a second ultrasound signal from the same ultrasound transducer of a second bandwidth, and wherein said second bandwidth is larger than said first bandwidth.

49. The computer-readable storage medium of paragraph 48, wherein said second bandwidth is at least two times larger than said first bandwidth.

50. The computer-readable storage medium of paragraph 48, wherein said second bandwidth is at least three times larger than said first bandwidth.

51. The computer-readable storage medium of paragraph 48, wherein said second bandwidth is at least four times larger than said first bandwidth.

52. The computer-readable storage medium of any of paragraphs 42-51, wherein said excitation portion of said second excitation waveform precedes said damping portion of said second excitation waveform.

53. The computer-readable storage medium of any of paragraphs 42-52, wherein said damping portion of said second excitation waveform is at least one of inverted and of a reduced amplitude compared to said excitation portion of said second excitation waveform.

54. The computer-readable storage medium of any of paragraphs 42-52, wherein said damping portion of said second excitation waveform comprises damping pulses of varying amplitude which are out of phase with said excitation portion of said second excitation waveform.

55. The computer-readable storage medium of any of paragraphs 42-52, wherein said damping portion of said second excitation waveform comprises an inverted cycle pulse of a first cycle with a smaller first amplitude than said excitation portion, followed by an inverted cycle pulse of a second cycle with a smaller second amplitude than said excitation portion, and wherein said second amplitude is also smaller than said first amplitude.

56. The computer-readable storage medium of paragraph 55, wherein said first cycle is a 1.5 cycle pulse and said second cycle is a 2 cycle pulse.

57. The computer-readable storage medium of any of paragraphs 42-52, wherein said dampening portion comprises a first dampening pulse of a first amplitude that is less than said excitation portion, a second dampening pulse following said first dampening pulse and that is of a second amplitude that is less than both said first amplitude and said excitation portion.

### BRIEF DESCRIPTION OF THE DRAWINGS

The subject matter of the present disclosure is particularly pointed out and distinctly claimed in the concluding portion of the specification. An understanding of the present disclosure may be further facilitated by referring to the following detailed description and claims in connection with the following drawings. While the drawings illustrate various embodiments employing the principles described herein, the drawings do not limit the scope of the claims.

FIG. 1 is a block diagram of an ultrasound system.

FIG. 2 is a diagram of an air-backed transducer that may be used by the ultrasound system of FIG. 1.

FIG. 3 is representative memory that may be used by the ultrasound system of FIG. 1 and that stores one or more excitation waveforms with no damping portion and that stores one or more excitation waveforms with both an excitation portion and a damping portion.

FIG. 4 is a protocol that may be stored in memory for execution by the ultrasound system of FIG. 1.

FIG. 5A illustrates representative excitation waveforms (1.5 cycle excitation) that may be provided to the ultrasound transducer of the ultrasound system of FIG. 1 (Example 1).

FIGS. 5B and 5C are outputs from the ultrasound transducer of the ultrasound system of FIG. 1, using the excitation waveforms of FIG. 5A (Example 1).

FIG. 5D illustrates representative excitation waveforms (1 cycle excitation) that may he provided to the ultrasound transducer of the ultrasound system of FIG. 1 (Example 1).

FIGS. 5E and 5F are outputs from the ultrasound transducer of the ultrasound system of FIG. 1, using the excitation waveforms of FIG. 5D (Example 1).

FIG. 6 is a schematic of a KLM model for generating simulated excitation waveforms that may be provided to an ultrasound transducer of an ultrasound system (Example 2).

FIG. 7 provides a listing of material properties for an ultrasound transducer in the model of FIG. 6 (Example 2).

FIG. 8 is a block diagram of an experimental setup for the model of FIG. 6 (Example 2).

FIG. 9A illustrates representative excitation waveforms (1.5 cycle excitation) that may be provided to the ultrasound transducer of the ultrasound system of FIG. 1 (Example 2).

FIGS. 9B and 9C are outputs from the ultrasound transducer of the ultrasound system of FIG. 1, using the excitation waveforms of FIG. 9A (Example 2).

FIG. 9D illustrates representative excitation waveforms (1 cycle excitation) that may be provided to the ultrasound transducer of the ultrasound system of FIG. 1 (Example 2).

FIGS. 9E and 9F are outputs from the ultrasound transducer of the ultrasound system of FIG. 1, using the excitation waveforms of FIG. 9D (Example 2).

FIG. 10A illustrates representative excitation waveforms (1.5 cycle excitation) that may be provided to the ultrasound transducer of the ultrasound system of FIG. 1 (Example 2).

FIGS. 10B and 10C are outputs from the ultrasound transducer of the ultrasound system of FIG. 1, using the excitation waveforms of FIG. 10A (Example 2).

FIG. 10D illustrates representative excitation waveforms (1 cycle excitation) that may be provided to the ultrasound transducer of the ultrasound system of FIG. 1 (Example 2).

FIGS. 10E and 10F are outputs from the ultrasound transducer of the ultrasound system of FIG. 1, using the excitation waveforms of FIG. 10D (Example 2).

### DETAILED DESCRIPTION

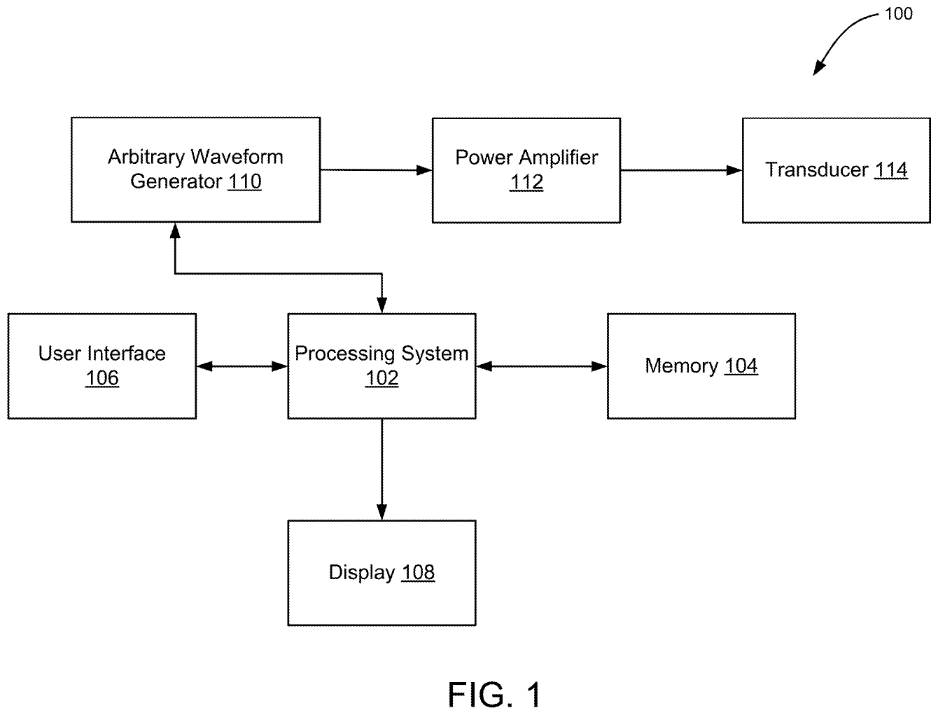

An ultrasound system is illustrated in FIG. 1 and is identified by reference numeral 100 (the receive circuitry and accompanying software not being illustrated in FIG. 1, since the present disclosure focuses on transmission from the ultrasound system 100). The ultrasound system 100 includes a processing system 102 (e.g., a central processing unit; one or more processors or microprocessors of any appropriate type and utilizing any appropriate processing architecture and including a distributed processing architecture), an arbitrary waveform generator 110, and an ultrasound transducer 114. The ultrasound transducer 114 may be of any appropriate type and/or configuration. An amplifier or a power amplifier 112 may be disposed between the arbitrary waveform generator 110 and the ultrasound transducer 114. A user interface 106 of any appropriate type (e.g., a monitor, a keyboard, a mouse, a touchscreen), memory 104, and a display 108 may each be operatively interconnected with the processing system 102. Although the user interface 106, processing system 102, memory 104, and display 108 are illustrated separately from the arbitrary waveform generator 110, it should be appreciated that one or more of these components (including all of these components) could actually be part of the arbitrary waveform generator 110.

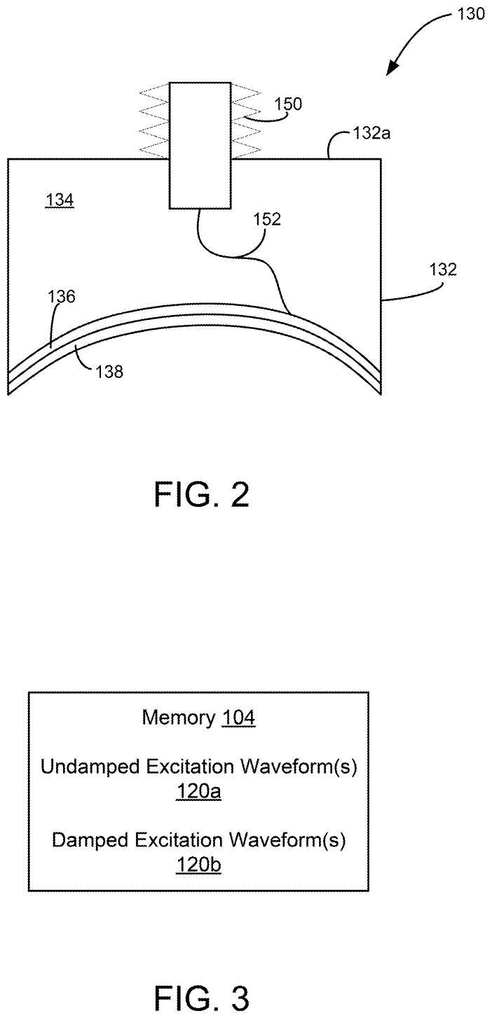

A representative transducer assembly that may be used by the ultrasound system 100 of FIG. 1 is illustrated in FIG. 2 and is identified by reference numeral 130. The transducer assembly 130 (or alternatively simply “transducer 130”) includes a housing 132 having a back wall 132a. A. transducer 136 (or alternatively a “transducer element 136” such as a piezo-electric component/layer) is oppositely disposed and spaced from the back wall 132a. A cavity 134 extends from the back wall 132a to the transducer 136. One or more matching layers 138 of any appropriate size, shape, and/or configuration may adjoin the transducer 136 externally of the cavity 134, although such a matching layer 138 may not be required in one or more instances. Note that the transducer assembly 130 of FIG. 2 excludes a backing layer.

A connector 150 (e.g., a coaxial connector) may be provided on the housing 132 (for instance, back wall 132a) to accommodate communication between the transducer assembly 130 and the arbitrary waveform generator 110. A wire or other conductor element 152 may extend from the connector 150 to the transducer 136 for purposes of transmitting an excitation waveform from the arbitrary waveform generator 110 to the ultrasound transducer 114.

The memory 104 may store a plurality of different excitation waveforms 120 that may be issued by the arbitrary waveform generator 110 and provided to the ultrasound transducer 114 as a drive signal. At least two different types of excitation waveforms 120 may be stored in the memory 104 and as shown in FIG. 3. One or more excitation waveforms 120a may be stored in the memory 104, with each of these excitation waveforms 120a including an excitation portion but no damping portion (e.g., an undamped excitation waveform 120a). One or more excitation waveforms 120b may be stored in the memory 104, with each of these excitation waveforms 120b including excitation portion and a damping portion (e.g., a damped excitation waveform 120b).

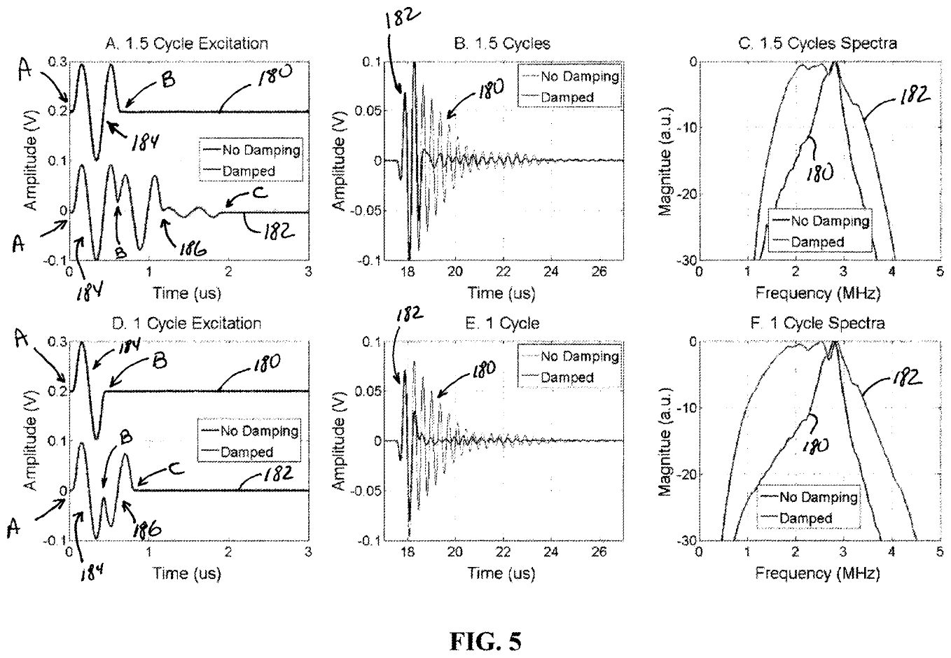

Excitation waveforms 120a may be characterized as being applicable to one or more ultrasound procedures (e.g., therapy), while excitation waveforms 120b may be characterized as being applicable to one or more different ultrasound procedures (e.g., imaging). Excitation waveforms 120a may be characterized as generating narrower bandwidths than are output from the ultrasound transducer 114 (compared to the excitation waveforms 120b), while excitation waveforms 120b may be characterized as generating wider or broader bandwidths than are output from the ultrasound transducer 114 (compared to the excitation waveforms 120a). Bandwidths that are output from the ultrasound transducer 114 using excitation waveforms 120b may be at least four times that of the bandwidths that are output from the ultrasound transducer 114 using excitation waveforms 120a in one or more embodiments. Bandwidths that are output from the ultrasound transducer 114 using excitation waveforms 120b may be at least three times that of the bandwidths that are output from the ultrasound transducer 114 using excitation waveforms 120a in one or more embodiments. Bandwidths that are output from the ultrasound transducer 114 using the excitation waveforms 120b may be at least two times that of the bandwidths that are output from the ultrasound transducer 114 using the excitation waveforms 120a in one or more embodiments. Excitation waveforms in accordance with excitation waveforms 120a and 120b from FIG. 3 are addressed in more detail below in relation to Examples 1 and 2, and including in relation to FIGS. 5, 9, and 10.

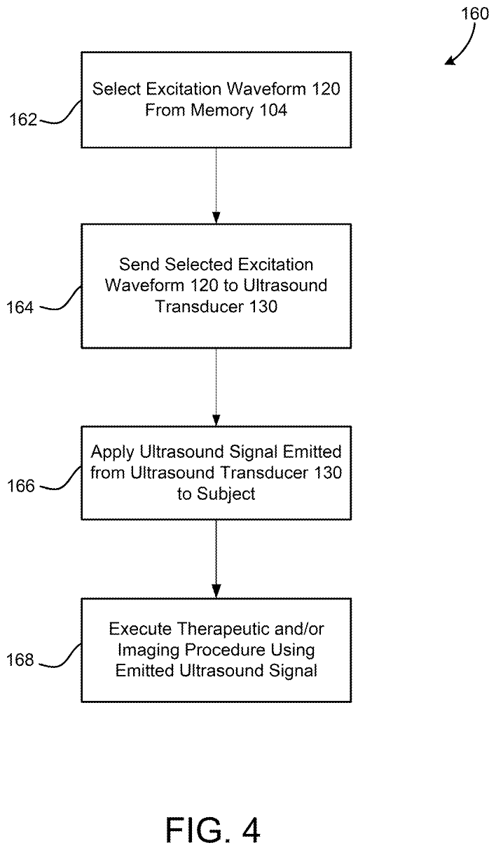

An embodiment of a protocol that may be stored in memory 104, executable by the processing system 102, and for outputting an excitation waveform 120 from the arbitrary waveform generator 110 for provision to/driving the ultrasound transducer 114 is illustrated in FIG. 4 and is identified by reference numeral 160. An excitation waveform 120 may be selected from the memory 104 (162). This selection may be done in any appropriate manner. For instance, multiple excitation waveforms 120 (e.g., one or more excitation waveforms 120a and one or more excitation waveforms 120b) may be presented on the display 108 for selection by a user through the user interface 106 of the ultrasound system 100 (FIG. 1). At least two different groups of excitation waveforms 120 could be presented on the display 108. One group could be a plurality of the first excitation waveforms 120a noted above, while another group could be a plurality of the second excitation waveforms 120b noted above.

The selected excitation waveform 120 (162) from the protocol of FIG. 4 is sent or transmitted by the arbitrary waveform generator 110 to the ultrasound transducer 130 (164). The excitation waveform 120 from the arbitrary waveform generator 110 excites the ultrasound transducer 114 and results in the emission of an ultrasound signal from the ultrasound transducer 114 to a subject such as a patient (166). This ultrasound signal may be used for any appropriate ultrasound procedure (168), such as for therapy, imaging, or the like.

In various embodiments, memory 104 is configured to store information used by the ultrasound system 100 (e.g., excitation waveforms 120a, 120b). In various embodiments, memory 104 comprises a computer-readable storage medium, which, in various embodiments, includes a non-transitory storage medium. In various embodiments, the term “non-transitory” indicates that the memory 104 is not embodied in a carrier wave or a propagated signal. In various embodiments, the non-transitory storage medium stores data that, over time, changes (e.g., such as in a random access memory (RAM) or a cache memory). In various embodiments, memory 104 comprises a temporary memory. In various embodiments, memory 104 comprises a volatile memory. In various embodiments, the volatile memory includes one or more of RAM, dynamic RAM (DRAM), static RAM (SRAM), and/or other forms of volatile memories. In various embodiments, memory 104 is configured to store computer program instructions for execution by the processing system 102 (e.g., protocol 160 of FIG. 4). In various embodiments, applications and/or software running on the processing system 102 utilize(s) memory 104 in order to temporarily store information used during program execution. In various embodiments, memory 104 includes one or more computer-readable storage media. In various embodiments, memory 104 is configured to store larger amounts of information than volatile memory. In various embodiments, memory 104 is configured for longer-term storage of information. In various embodiments, memory 104 includes non-volatile storage elements, such as, for example, electrically programmable memories (EPROM), electrically erasable and programmable (EEPROM) memories, flash memories, floppy discs, magnetic hard discs, optical discs, and/or other forms of memories.

In various embodiments, the processing system 102 is configured to implement functionality and/or process instructions. In various embodiments, the processing system 102 is configured to process computer instructions stored in memory 104 (e.g. to execute protocol 160 of FIG. 4). In various embodiments, the processing system 102 includes one or more of a microprocessor, a controller, a digital signal processor (DSP), an application specific integrated circuit (ASIC), a field-programmable gate array (FPGA), or other equivalent discrete or integrated logic circuitry. In various embodiments, display 108 comprises one or more of a screen, touchscreen, or any other suitable interface device(s) that is configured to allow a user to interact and control the imaging system 100 (e.g., at least part of the user interface 106 could be combined with. the display 108).

System program instructions and/or processor instructions may be loaded onto memory 104. The system program instructions and/or processor instructions may, in response to execution by operator, cause the processing system 102 to perform various operations and including the execution of the protocol 160 of FIG. 4. The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In re Nuijten to fall outside the scope of patentable subject matter under 35 U.S.C. § 101.

### EXAMPLE 1

A modified 1-D transmission line model was written in Matlab to accommodate arbitrary waveform excitation. Excitation waveforms designed to dampen subsequent ringing of air-backed transducers were optimized through a design of experiments to achieve the broadest bandwidth possible. These excitation waveforms consist of a 1 cycle or 1.5 cycle excitation pulse followed by an inverted 1 or 1.5 cycle dampening pulse with reduced amplitude. This was experimentally verified using a 3 MHz, 26 mm diameter, spherically focused, air-backed transducer. Waveforms similar to waveforms created in the model were programmed into an arbitrary waveform generator whose output served as the input into a power amplifier. A hydrophone was placed at the focal point to capture the emitted pulse waveform.

FIG. 5 shows initial hydrophone results from 1.5-cycle (top row—FIGS. 5B and 5C) and 1-cycle (bottom row—FIGS. 5E and 5F) excitation. In all graphs of FIG. 5, traces 180 are associated with an excitation portion 184 without a subsequent damping portion 186 in the waveform provided to the transducer (FIGS. 5A and 5D), and traces 182 are associated with the use of an excitation portion 184 followed by a damping portion 186 in the waveform provided to the transducer (FIGS. 5A and 5D). The left column (FIGS. 5A and 5D) shows the excitation waveforms provided to the transducer. The trace 180 of FIG. 5A (1.5 cycle) includes an excitation portion 184 without a subsequent damping portion 186. The trace 182 of FIG. 5A (1.5 cycle) includes an excitation portion 184 (from A to B), followed by a damping portion 186 (from B to C). The trace 180 of FIG. 5D (1 cycle) includes an excitation portion 184 without a subsequent damping portion 186. The trace 182 of FIG. 5D (1 cycle) includes an excitation portion pulse 184 (from A to B), followed by a damping portion 186 (from B to C). In FIG. 5A, a vertical offset is added to the no damping case for clarity. FIGS. 5B/5E and 5C/5F each illustrate the output from the transducer.

For 1.5-cycle excitation and as shown in FIG. 5C, the −3 dB bandwidth increased from 10.1% with 2.74 MHz center frequency (trace 180—no damping) to 44.0% at 2.40 MHz center frequency (trace 182—damping). For 1-cycle excitation and as shown in FIG. 5F, the −3 dB bandwidth increased from 11.4% at 2.73 MHZ (trace 180—no damping) to 63.8% at 2.26 MHz (trace 182 damping). In both cases, the peak-to-peak amplitude in the damped case is comparable to the undamped case. When damping is applied, low amplitude ringing is still observed. This ringing may be suppressed through further optimization and electrical tuning. This technique could be applied to other unconventional transducer designs such as dual-frequency and dual-layer transducers.

### EXAMPLE 2

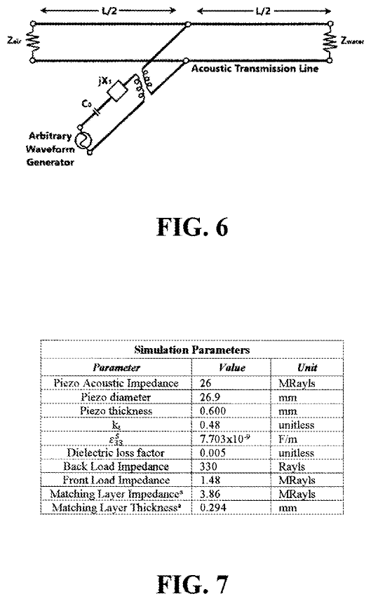

A 1-D KLM transmission line model was implemented in Matlab (Natwick, Mass.). The KLM model is a frequency domain model for transducers. A schematic of a modified KLM model is shown in FIG. 6. Values for Co, X₁, and the transformer turns ratio φ are calculated. using equations given by KLM. L is the thickness of the PZT (e.g., the transducer). The PZT material used is DL-47 from DeL Piezo Specialties, LLC (West Palm Beach, USA) with its material properties being listed in FIG. 7. The Matlab implementation uses a transmission or T-matrix approach where each circuit element was modeled by a 2×2 matrix. To adapt the KLM model for arbitrary waveform generation, arbitrary waveforms are generated first in the time domain. The arbitrary waveforms consist of an initial excitation pulse followed by damping pulses of varying amplitude which are out of phase with respect to the excitation pulse. The discrete Fourier Transform of the waveform is performed and used as the drive spectrum in the KLM model. In this work, 1 cycle and 1.5 cycle excitations were used. Arbitrary waveforms were empirically and iteratively created to achieve the broadest bandwidth. These waveform were created by adjusting the amplitude of each subsequent pulse. Further optimization of the bandwidth was subsequently performed using a multidimensional unconstrained nonlinear minimization (Nelder-Mead) in Matlab. The modified KLM model was set up as an objective function whose output to be minimized was the ripple energy after the main pulse. In these optimizations, the ripple energy included all signal beyond the 1-cycle or 1.5-cycle excitation. The empirically determined amplitudes of the damping pulses were used as a starting point for the minimization. In the minimization process, energy in the ripple after the main pulse served as the function output to be minimized. The minimization process was limited to a maximum of 10,000 iterations which took approximately 3 minutes to complete using a 2015 Macbook Pro laptop. In this first simulation, the transmit waveform was optimized using an air-backed transducer with no matching layer.

In a second simulation, a single quarter-wave matching layer was added, and the optimization process was repeated. Iterative and empirical adjustments were first made to the damping pulses, and further optimization was performed using multidimensional unconstrained nonlinear mimization using the same ripple criteria as in the no matching layer case. The acoustic impedance of the matching layer, Z_{ML}, was 3.86 MRalys as given by the equation

Z_{ML}=Zₚ^{⅓}Zₜ^{⅔}

where Zₚ is the acoustic impedance of the piezoelectric material and Zₜ is the acoustic impedance of the front medium. The thickness of the matching layer was set to 0.294 mm. Lastly, the performance of active damping with arbitrary waveform generators was simulated in a pulse-echo scenario using a single matching layer.

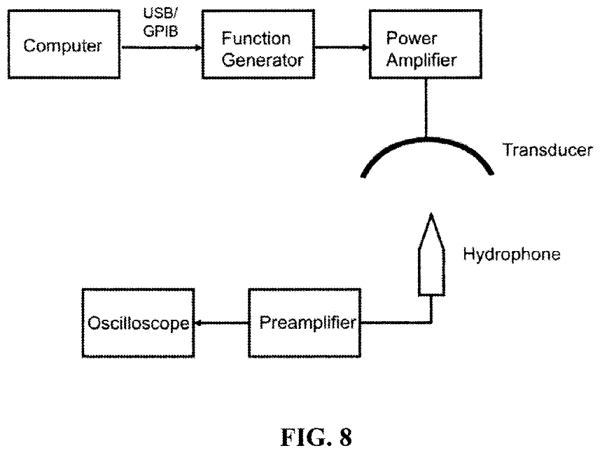

Arbitrary waveforms were created in Matlab and downloaded to a Tektronix AFG2020 Agilent function generator whose output served as the input into the ENI power amplifier. A spherically focused, air-backed 26 mm diameter PZT (DL. 47) transducer was connected to the output of ENI power amplifier. This transducer had a focal spot at 26 mm depth. This transducer had no matching layer. An Onda AGL-2020 hydrophone was placed at the focal point to record the acoustic output. Hydrophone recordings were captured using a Tektronix oscilloscope. The data was then imported in to Matlab for subsequent spectral analysis. FIG. 8 shows the experimental setup.

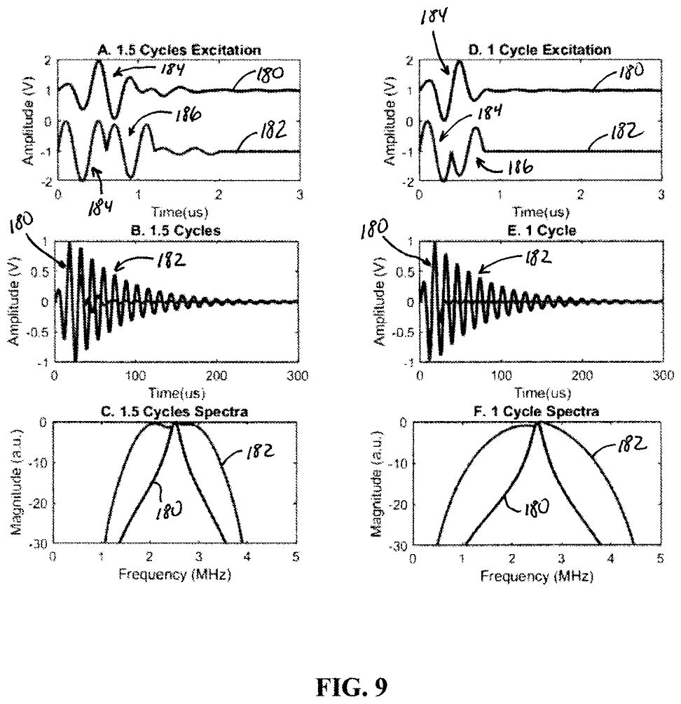

FIG. 9 shows simulated results from the modified KLM model for 1.5 cycle excitation and 1 cycle excitation using an air-backed transducer with no matching layer. FIGS. 9A and 9D show the corresponding waveform provided to the transducer, while FIGS. 9B/9E and 9C/9F illustrate the output from the transducer. The trace 180 of each of FIGS. 9A and 9D include an excitation portion 184 but no damping portion 186, while the trace 182 of each of FIGS. 9A and 9D include an excitation portion 184 followed by a damping portion 186. The traces 180 of FIGS. 9B/9E and FIGS. 9C/9F (all outputs from the transducer) are for the waveforms provided to the transducer with only an excitation portion 184 (FIGS. 9A and 9D), while the traces 182 of FIGS. 9B/9E and FIGS. 9C/9F (all outputs from the transducer) are for the waveforms provided to the transducer having an excitation portion 184 followed by a damping portion 186 (FIGS. 9A and 9D).

For 1 cycle excitation and as shown in FIG. 9F, the −3 dB bandwidth increases from 7.12% (no damping—trace 180) to 56.9% (damping—trace 182) and the center frequency without damping (trace 180) is 2.51 MHz while the center frequency with damping is 2.45 MHz (trace 182). To achieve this increase in bandwidth, an inverted 1-cycle pulse with relative amplitude 0.799 was used as the damping portion 186 (FIG. 9D). For 1.5-cycle excitation and as shown in FIG. 9C, the −3 dB bandwidth increases from 7.13% (no damping—trace 180) to 54.3% (damping—trace 182). To achieve this increase in bandwidth, an inverted 1.5-cycle pulse with relative amplitude of 0.89 was used followed by a 2-cycle pulse with relative amplitude of 0.1 (damping portion 186—FIG. 9A). These amplitudes are relative to the amplitude of the initial excitation signal. Data pertaining to 1.5 cycle excitation is shown in the left column of FIG. 9 (FIGS. 9A-9C), and data pertaining to 1 cycle excitation is shown in the right column of FIG. 9 (FIGS. 9D-9F).

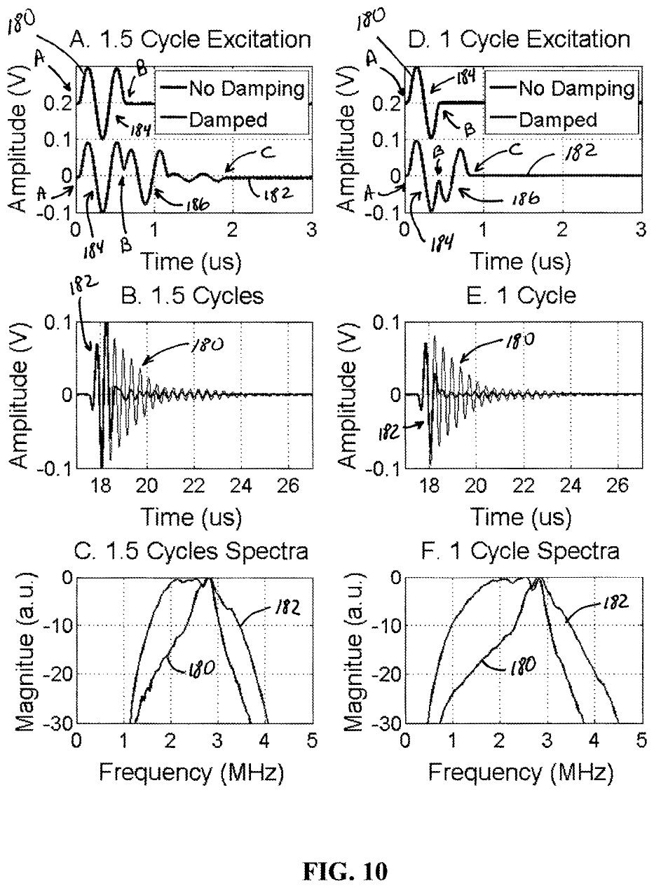

FIG. 10 shows experimental results using an air-backed transducer with no matching layer. FIGS. 10A and 10D show the corresponding waveform provided to the transducer, while FIGS. 10B/10E and 10C/10F illustrate the output from the transducer. The trace 180 of each of FIGS. 10A and 10D include an excitation portion 184 (A to B) but no damping portion 186, while the trace 182 of each of FIGS. 10A and IOD include an excitation portion 184 (A to B) followed by a damping portion 186 (B to C). The traces 180 of FIGS. 10B/10E and FIGS. 10C/10F (outputs from the transducer) are for the waveforms provided to the transducer with only an excitation portion 184 (FIGS. 10A and 10D), while the traces 182 of FIGS. 10B/10E and FIGS. 10C/10F (outputs from the transducer) are for the waveforms provided to the transducer having an excitation portion 184 followed by a damping portion 186 (FIGS. 10A and 10D).

The top row of FIG. 10 shows the excitation waveforms for 1.5 cycle (left—FIG. 10A) and 1 cycle (right—FIG. 10D). For 1.5 cycle, the out-of-phase damping pulses consisted of two 1.5 cycle pulses (damping portion 186 of trace 182—from B to C). The amplitude of the first dampening pulse (damping portion 186 of trace 182) was 77.5% of the initial excitation pulse (excitation portion 184 of trace 182—A to B), and the amplitude of the second dampening pulse (damping portion 186 of trace 182) was 10% of the initial excitation pulse (excitation portion 184 of trace 182—A to B). For 1 cycle excitation, the dampening pulse (damping portion 186 of trace 182 from B to C) consisted of a single out-of-phase cycle with amplitude of 75% of the initial excitation pulse (excitation portion 184 of trace 182—A to B). In FIGS. 10A and 10D, a vertical offset is added to the no damping case (trace 180) for clarity. For 1.5-cycle excitation and as illustrated in FIG. 10C, the −3 dB bandwidth increased from 10.1% with 2.74 MHz center frequency (no damping—trace 180) to 44.0% at 2.4 MHz center frequency (damping—trace 182). For 1-cycle excitation and as illustrated in FIG. 10F, the −3 dB bandwidth increased from 11.4% at 2.73 MHz (no damping—trace 180) to 63.8% at 2.26 MHz (damping−trace 182). In both cases, the peak-to-peak amplitude in the damped case is comparable to the undamped case. When damping is applied, low amplitude ringing is still observed.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

Any feature of any other various aspects addressed in this disclosure that is intended to be limited to a “singular” context or the like will be clearly set forth herein by terms such as “only,” “single,” “limited to,” or the like. Merely introducing a feature in accordance with commonly accepted antecedent basis practice does not limit the corresponding feature to the singular. Moreover, any failure to use phrases such as “at least one” also does not limit the corresponding feature to the singular. Use of the phrase “at least substantially,” “at least generally,” or the like in relation to a particular feature encompasses the corresponding characteristic and insubstantial variations thereof (e.g., indicating that a surface is at least substantially or at least generally flat encompasses the surface actually being flat and insubstantial variations thereof). Finally, a reference of a feature in conjunction with the phrase “in one embodiment” does not limit the use of the feature to a single embodiment.

What is claimed is:

1. An ultrasound system, comprising:
an arbitrary waveform generator;
an ultrasound transducer operatively connected with said arbitrary waveform generator; and
memory comprising a plurality of excitation waveforms transmittable from said arbitrary waveform generator to said ultrasound transducer, wherein a first excitation waveform of said plurality of excitation waveforms comprises an excitation portion with no damping portion, and wherein a second excitation waveform of said plurality of excitation waveforms comprises an excitation portion and a damping portion.

2. The ultrasound system of claim 1, wherein said ultrasound transducer comprises an air-backed ultrasound transducer.

3. The ultrasound system of claim 1, wherein said ultrasound transducer excludes a backing layer.

4. The ultrasound system of claim 1, further comprising a plurality of said first excitation waveforms.

5. The ultrasound system of claim 4, wherein each of said plurality of first excitation waveforms uses a different said excitation portion.

6. The ultrasound system of claim 1, further comprising a plurality of said second excitation waveforms.

7. The ultrasound system of claim 6, wherein each of said plurality of second excitation waveforms uses a different said excitation portion, a different said damping portion, or a combination thereof.

8. The ultrasound system of claim 1, wherein said first excitation waveform output from said arbitrary waveform generator outputs a first ultrasound signal from said ultrasound transducer of a first bandwidth, wherein said second excitation waveform output from said arbitrary waveform generator outputs a second ultrasound signal from said ultrasound transducer of a second bandwidth, and wherein said second bandwidth is larger than said first bandwidth.

9. The ultrasound system of claim 8, wherein said second bandwidth is at least three times larger than said first bandwidth.

10. The Ultrasound system of claim 1, wherein said excitation portion of said second excitation waveform precedes said damping portion of said second excitation waveform.

11. The ultrasound system of claim 1, wherein said damping portion of said second excitation waveform is at least one of inverted and of a reduced amplitude compared to said excitation portion of said second excitation waveform.

12. The ultrasound system of claim 1, wherein said damping portion of said second excitation waveform comprises damping pulses of varying amplitude which are out of phase with said excitation portion of said second excitation waveform.

13. The ultrasound system of claim 1, wherein said damping portion of said second excitation waveform comprises an inverted cycle pulse of a first cycle with a smaller first amplitude than said excitation portion, followed by an inverted cycle pulse of a second cycle with a smaller second amplitude than said excitation portion, and wherein said second amplitude is also smaller than said first amplitude.

14. The ultrasound system of claim 13, wherein said first cycle is a 1.5 cycle pulse and said second cycle is a 2 cycle pulse.

15. The Ultrasound system of claim 1, wherein said dampening portion comprises a first dampening pulse of a first amplitude that is less than said excitation portion, a second dampening pulse following said first dampening pulse and that is of a second amplitude that is less than both said first amplitude and said excitation portion.

16. The ultrasound system of claim 1, further comprising at least one of a user interface and a display.

17. A method of executing an ultrasound procedure using an ultrasound system comprising an arbitrary waveform generator, memory, and an ultrasound transducer, said method comprising:
selecting an excitation waveform from a plurality of excitation waveforms stored in said memory and that defines a selected excitation waveform, wherein a first excitation waveform of said plurality of excitation waveforms comprises an excitation portion with no damping portion, and wherein a second excitation waveform of said plurality of excitation waveforms comprises an excitation portion and a damping portion;
sending said selected excitation waveform from said arbitrary waveform generator to said ultrasound transducer; and
transmitting an ultrasound signal from said ultrasound transducer in response to said sending.

18. The method of claim 17, further comprising:
presenting said plurality of excitation waveforms on a display, wherein said selecting comprises using a user interface to select one of said plurality of excitation waveforms presented on said display.

19. The method of claim 18, wherein said presenting comprises presenting said plurality of excitation forms in two different groups, wherein a first group comprises a plurality of said first excitation waveforms, and wherein a second group comprises a plurality of said second excitation waveforms.

20. A computer-readable storage medium, comprising:
a plurality of excitation waveforms, wherein a first excitation waveform of said plurality of excitation waveforms comprises an excitation portion with no damping portion, and wherein a second excitation waveform of said plurality of excitation waveforms comprises an excitation portion and a damping portion; and
a protocol configured to:
present at least some of said plurality of excitation waveforms on a display; and
allow for selection of any one of said plurality of excitation waveforms through a user interface.
